(19) United States
(12) Patent Application Publication
(10) Pub. No.: US 2023/0002315 A1
(43) Pub. Date: Jan. 5, 2023

(54) Processes for the Preparation of Alpha-Hydroxy Esters via Grignard Coupling and Thiolation Reactions

(71) Applicant: Kemin Industries, Inc., Des Moines, IA (US)

(72) Inventors: Shengshu Huang, Zhuhai City (CN); Fangyi Li, Tianjin City (CN); Li Xu, Xi'an City (CN); Filip Nuyens, Geel (BE); Ye Lao, West Des Moines, IA (US); Jatuporn Salaklang, Mol (BE)

(73) Assignee: Kemin Industries, Inc., Des Moines, IA (US)

(21) Appl. No.: 17/777,402

(22) PCT Filed: Nov. 19, 2020
(86) PCT No.: PCT/US2020/061242

§ 371 Date: May 17, 2022

(30) Foreign Application Priority Data
Nov. 22, 2019 (CN) PCT/CN2019/120393

(51) Int. Cl. C07C 319/18; C07C 69/738; C07C 323/52; C07C 67/08; A23K 20/105; A23K 50/10

(52) U.S. Cl. CPC C07C 319/18; C07C 69/738; C07C 323/52; C07C 67/08; A23K 20/105; A23K 50/10

(57) ABSTRACT

The present disclosure provides processes for preparing an alpha-hydroxy ester by addition of a vinyl Grignard reagent to an oxalate ester and thiolation of the resulting double bond. Also provided are alpha-hydroxy esters and synthetic intermediates prepared according to processes disclosed herein and compositions comprising the alpha-hydroxy esters.

### RELATED APPLICATIONS

This application claims priority to International Application No. PCT/CN2019/120393 filed Nov. 22, 2019, which is incorporated by reference herein in its entirety for any purpose.

### FIELD OF THE INVENTION

The present disclosure provides processes for preparing an alpha-hydroxy ester by addition of a vinyl Grignard reagent to an oxalate ester and thiolation of the resulting double bond. Also provided are alpha-hydroxy esters and synthetic intermediates prepared according to processes disclosed herein, compositions comprising the alpha-hydroxy esters, and methods of using the compositions.

### BACKGROUND

Alpha-hydroxy ester analogs of natural amino acids are useful as dietary supplements and in the study of enzymatic processes and protein function. Synthesis of such esters typically employs acid-catalyzed Fischer esterification of the corresponding acid and an alcohol in the presence of a strong acid such as H₂SO₄ or Amberlyst® cationic exchange resin, acid-mediated hydrolysis of the corresponding nitrile in the presence of a strong acid, or enzyme-mediated processes. However, acid-catalyzed approaches lead to degradation of starting material and product and contamination of the product with dimeric and oligomeric components. Such methods typically provide low yields, and complex purification techniques are needed to isolate the target compound from the polymeric side-products. Enzymatic approaches require expensive and sensitive reagents and special reaction conditions.

An alpha-hydroxy ester of particular importance is isopropyl 2-hydroxy-4-(methylthio)butanoate (HMBi). HMBi is the isopropyl ester of the hydroxy analog of methionine, 2-hydroxy-4-(methylthio)butanoic acid (HMBA). HMBi is used to help supplement methionine in ruminants, including cows. Adequate methionine levels in dairy cows help maintain desired levels of milk protein synthesis and, in turn, desired levels of milk production. However, methionine content in the animal feedstock is vastly insufficient and has become a major limiting factor in the diet of the dairy cow. HMBi is a chemical derivative of methionine that readily and rapidly diffuses through the rumen wall, avoiding degradation by ruminal microbes. Once HMBi passes through the rumen wall, it is metabolized in the liver and becomes available for milk protein synthesis in dairy cows.

There is a need for additional processes for synthesizing alpha-hydroxy esters, such as HMBi, that employ inexpensive, non-toxic reagents and mild reaction conditions, and that provide the product esters in high yield and purity.

### SUMMARY

In one aspect, the disclosure is directed to a method of preparing a compound of Formula (I):

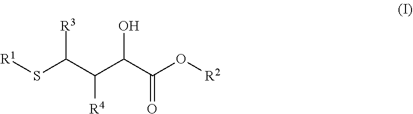

wherein
R¹ is C₁₋₄ alkyl; and
R² is C₁₋₈ alkyl or C₄₋₇ cycloalkyl; and
R³ and R⁴ are each independently chosen from H, methyl, and ethyl;
comprising coupling a compound of Formula (IV):

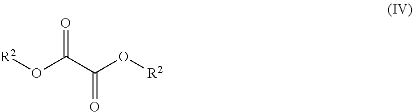

with a vinyl Grignard reagent of Formula (A):

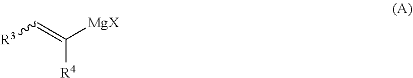

wherein X is Br or Cl;
to form a compound of Formula (III):

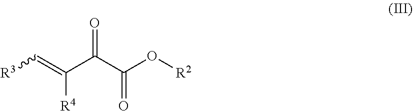

and converting the compound of Formula (III) to the compound of Formula (I).

In another aspect, the disclosure is directed to a method of preparing a compound of Formula (I), comprising reducing a compound of Formula (II):

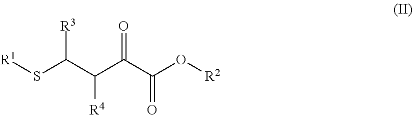

with a reducing agent to form the compound of Formula (I).

In some aspects, the compound of Formula (I) is the compound of Formula (I-A):

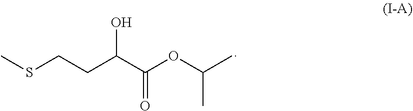

In another aspect, the disclosure is directed to a method of preparing a compound of Formula (I-A):

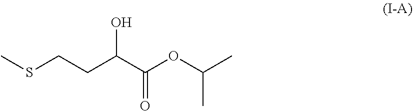

comprising:
esterifying oxalic acid with isopropanol to form diisopropyl oxalate;
coupling diisopropyl oxalate with vinylmagnesium bromide to form a compound of Formula (III-A):

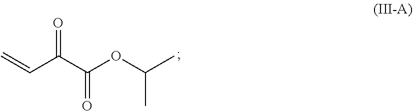

thiolating the compound of Formula (III-A) with CH₃SH to form a compound of Formula (II-A):

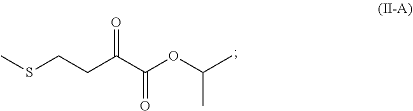

and reducing the compound of Formula (II-A) to form the compound of Formula (I-A).

In another aspect, the disclosure is directed to a compound of Formula (I) or Formula (I-A) prepared as in any of the methods described herein.

In another aspect, the disclosure is directed to isopropyl 2-oxobut-3-enoate.

In another aspect, the disclosure is directed to an animal feed composition comprising the compound of Formula (I) of Formula (I-A) as described herein. In some aspects, the animal feed is a cow feed, such as a dairy cow feed.

In another aspect, the disclosure is directed to a method of supplying bioavailable methionine to a dairy cow comprising administering to the cow a compound of Formula (I) or Formula (I-A) or an animal feed composition as described herein. In another aspect, the disclosure is directed to a method of supplying at least about 50% bioavailable methionine to a dairy cow comprising administering to the cow a compound of Formula (I) or Formula (I-A) or an animal feed composition as described herein. In another aspect, the disclosure is directed to a method of improving milk obtained from a dairy cow, comprising supplying to the cow a compound of Formula (I) or Formula (I-A) or an animal feed composition as described herein.

In another aspect, the disclosure is directed to a method of improving the condition of a cow comprising supplying to the cow a compound of Formula (I) or Formula (I-A) or an animal feed composition as described herein.

### BRIEF DESCRIPTION OF THE FIGURES

FIG. 1A is a ¹³C NMR spectra of diisopropyl oxalate, as described in Example 1.

FIG. 1B is a ¹H NMR spectra of diisopropyl oxalate, as described in Example 1.

FIG. 2A is a ¹³C NMR spectra of 2-oxo-4-methylthiobutanoic acid isopropyl ester, as described in Example 3.

FIG. 2B is a ¹H NMR spectra of 2-oxo-4-methylthiobutanoic acid isopropyl ester, as described in Example 3.

FIG. 3A is a ¹³C NMR spectra of 2-hydroxy-4-methylthiobutanoic isopropyl ester (HMBi), as described in Example 5.

FIG. 3B is a ¹H NMR spectra of 2-hydroxy-4-methylthiobutanoic isopropyl ester (HMBi), as described in Example 5.

### DETAILED DESCRIPTION

Unless otherwise stated, the terms in this disclosure carry their plain and ordinary meaning as understood by those in the relevant art. The following terms used in the specification and claims are defined for the purposes of this disclosure and have the following meanings.

As used herein, the terms “isopropyl 2-hydroxy-4-(methylthio)butanoate,” “HMBi,” and “isopropyl ester of 2-hydroxy-4-(methylthio)butanoic acid” refer to a compound of the following structure (Formula (I), where R¹ is methyl and R² is isopropyl, shown below as Formula (I-A)).

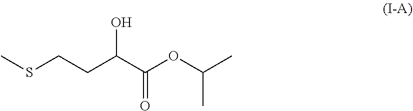

As used herein, the terms “2-hydroxy-4-(methylthio)butanoate,” “2-hydroxy-4-(methylthio)butanoic acid,” and “HMBA” refer to a compound of the following structure.

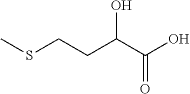

The compounds described herein may exist in racemic form, as a single enantiomer, or as a mixture of enantiomers. Thus, for example, HMBi refers to racemic HMBi (or “DL-HMBi”), or to D-HMBi or L-HMBi, or a mixture thereof.

Compounds described herein may also exist in salt forms. Chemical formulae shown herein should be understood to include the structures shown as well as salt forms thereof. For example, where a compound includes a carboxylic acid, the formula also encompasses salt forms of the conjugate base (carboxylate), such as sodium, potassium, magnesium, or calcium salts. Where a compound includes an indole or imidazole group, the formula also encompasses salt of the conjugate acids thereof, such as HCl salts.

“Alkyl” means a linear saturated monovalent hydrocarbon radical of one to eight carbon atoms (for example, one to six carbon atoms, one to four carbon atoms, or one to three carbon atoms) or a branched saturated monovalent hydrocarbon radical of three to eight carbon atoms (for example, three to six carbon atoms, three to four carbon atoms, or three carbon atoms), e.g., methyl, ethyl, propyl, isopropyl, butyl, isobutyl, sec-butyl, tert-butyl, pentyl (including all isomeric forms), and the like.

“Cycloalkyl” means a cyclic saturated monovalent hydrocarbon radical of three to ten carbon atoms, e.g., cyclopropyl, cyclobutyl, cyclopentyl, or cyclohexyl, and the like.

“Optional” or “optionally” means that the subsequently described event or circumstance may but need not occur, and that the description includes instances where the event or circumstance occurs and instances in which it does not. For example, an alkyl group “optionally substituted with —OH” means that the —OH may but need not be present, and the description includes situations where the alkyl group is substituted with an —OH group and situations where the alkyl group is not substituted with an —OH group.

The term “reaction solvent” refers to an organic liquid that is used to carry dissolved reactants. In some embodiments, one of the reagents of the reaction serves as a reagent and as the reaction solvent. In other embodiments, the reagents are diluted in a different reaction solvent.

The term “acid catalyst” refers to an acid added to a reaction in a sub-stoichiometric amount that serves to catalyze the reaction. An acid catalyst may be a Bronsted acid (such as an acid with a pKa of less than 7, such as HCl, H₂SO₄, KHSO₄, acetic acid, and the like) or a Lewis acid (such as boronic acid). In some embodiments, the acid is generated in situ, e.g., by reaction of acetyl chloride or TMSCl with water or an alcohol.

The term “concentration” refers to the amount of solute in a solvent. Herein, concentrations may be depicted by weight % or by molarity (M) or normality (N).

The term “heptane” or “n-heptane” refers to pure n-heptane, or n-heptane in a mixture with other C7 isomers (e.g., at least 90% n-heptane and at least 95% total C7 isomers).

The term “reflux temperature” or “reflux” refers to the temperature at which a reaction solvent boils; typically, a condenser is used to cool the solvent vapor and condense it back into the reaction vessel. The precise temperature at which a given solvent reaches reflux may vary depending on environmental factors.

The term “about” refers to a numeric value, including, for example, whole numbers, fractions, and percentages, whether or not explicitly indicated. The term about generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded.

The term “extract,” “extraction,” or “extracting,” refers to a process of partitioning a material between an organic phase and an aqueous phase. In some aspects, the extracting is performed on a reaction mixture or a concentrated residue of a reaction mixture. An “extract” is the organic phase once separated from the aqueous phase. As used herein, extracting does not encompass purification methods performed on a crude reaction product, such as simple distillation, vacuum distillation, azeotropic distillation, fractional distillation, continuous distillation, flash chromatography, HPLC, or recrystallization.

As used herein, “purification” or “purifying” refers to a method of isolating the product of a reaction following completion of the reaction. Purification methods include simple distillation, vacuum distillation, azeotropic distillation, fractional distillation, continuous distillation, flash chromatography, HPLC, or recrystallization.

The term “substantially,” e.g., “substantially in monomeric form” refers to the purity of a compound of Formula (I), relative to dimeric and/or oligomeric analogs.

As used herein, the term “dimer” or “dimeric compound” refers to a compound in which two molecules of a given monomer structure, or one molecule each of two different monomer structures, are condensed into a single molecule. As used herein, the term “oligomer” or “oligomeric compound” refers to a compound in which more than two molecules of a given monomer structure, or more than two molecules of at least two different monomer structures, are condensed into a single polymeric structure. HMBi may form homogeneous oligomers or heterogeneous HMBi oligomers (comprising at least one HMBi monomer unit).

The term “purity” or the expression of a percentage compound (e.g., x % HMBi) refers to the purity of a compound in a sample, as determined by weight, by GC analysis, and/or by HPLC analysis. In some aspects, the purity by weight is determined by GC or HPLC analysis with UV detection.

The term “purity by weight” refers to the purity of a compound in a sample with respect to other components in the sample, where the ratio of the mass of the compound to the mass of the sample is expressed as a percentage.

The term “purity” with reference to gas chromatography (GC) or HPLC purity means the calculated purity (expressed in %) of the peak area for the compound of interest relative to the sum of all the peak areas in the chromatogram. In some aspects, purity is determined by HPLC with UV detection.

In some aspects, purity is the purity required according to marketing regulations for a regulated product. In the case of HMBi, for example, the compound comprises 0.5% water or less (e.g., as determined by Karl-Fischer analysis). (See Commission Implementing Regulation (EU) No 469/2013 of 22 May 2013.)

The terms “crude,” “crude product,” and “crude compound” refer to the sample of a compound obtained from a reaction mixture after concentration of the reaction mixture and/or extraction of the reaction mixture into an organic solvent and concentration of the organic extract.

The term “animal feed composition” refers to a product suitable for use in animal nutrition. In some aspects, the animal feed composition is an animal feed (e.g., food or drinking water comprising the supplement), and in some aspects, the animal feed composition is a feed additive. The feed additive is suitable for mixing with animal feedstuff or with drinking water.

The term “carrier” refers to a suitable carrier for an animal feed additive. Suitable carriers include water (for a liquid or solid feed additive) or silica (for a solid feed additive). In some aspects, the carrier is silica (silicon dioxide). In some aspects, the feed additive comprises the compound and silica in a 3:2 ratio.

In some aspects, an animal feed comprises a pelleted, protein-rich feed (e.g., based on groundnuts, rape seed meal, and/or soybean meal) supplemented with 2.5% or 1% HMBi by weight. In some aspects, an animal feed comprises about 45% and about 50% cereal (maize, barley, wheat, and/or wheat by-products), supplemented with 0.5% or 3.0% HMBi by weight. In some aspects, an animal feed comprises a mash feed with molasses, or a pelleted feed, each supplemented with 2.5% or 1% HMBi by weight.

The term “administering” refers to providing the supplement to the target animal. Administering may be done orally, e.g., through ingestion of food or drinking water comprising the compound, or by injection or other mode of administration.

As used herein, “improving milk” refers to an improvement in the quality and/or quantity of milk produced by a treated cow or a group of treated cows as compared to that produced by untreated counterparts. Improvements in milk include, for example, increased protein content in the milk (e.g., increase in alpha, beta, and/or kappa proteins), increased fat content in the milk, and/or increased volume of milk produced.

As used herein, “improving the condition of a cow” refers to an improvement in a health measure of treated cow or group of treated cows as compared to the health measure in untreated counterparts. Improvement of the condition of a cow can refer to, for example, an increase in some characteristic relative to untreated animal; e.g., weight gain.

As used herein, an “improvement in fertility” includes, for example, shortening the interval between calving and reproduction and/or increasing the percentage fertilization during insemination.

As used herein, an “improvement in liver function” includes, for example, reduction in metabolic problems, improvement in levels of very low-density lipoproteins, reduction in blood ketosis, and/or reduction in the incidence of hepatic steatosis.

As used herein, “increase in energy” refers to, for example, stimulation of fermentation processes in the rumen, resulting in an increase in digestible organic matter, and therefore more energy for the animal.

### Synthetic Processes

The disclosure relates to methods of preparing a compound of Formula (I) or Formula (I-A) and/or an intermediate through one or more of the following reactions: a) esterification of oxalyl chloride or oxalic acid with R²—OH to form an oxalate diester; b) coupling the oxalate diester with an alkenyl Grignard reagent to form an alkenyl-substituted alpha-keto ester (a 2-oxobut-3-enoate ester); c) thiolation of the alkenyl-substituted alpha-keto ester to form a 4-alkylthio-2-oxo-butanoate ester; and d) reduction of the 4-alkylthio-2-oxo-butanoate ester to form the compound of Formula (I) or Formula (I-A). Oxalic acid may used as, for example, oxalic acid or oxalic acid dihydrate.

In some embodiments, the disclosure relates to method of preparing a compound of Formula (I):

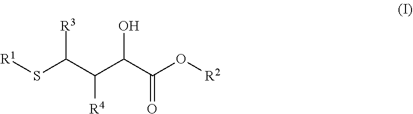

wherein
R¹ is C₁₋₄ alkyl; and
R² is C₁₋₈ alkyl or C₄₋₇ cycloalkyl; and
R³ and R⁴ are each independently chosen from H, methyl, and ethyl;
comprising coupling a compound of Formula (IV):

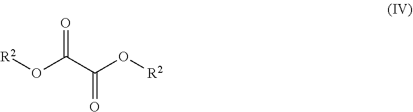

with a vinyl Grignard reagent of Formula (A):

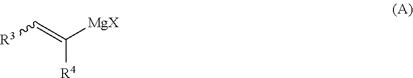

wherein X is Br or Cl;
to form a compound of Formula (III):

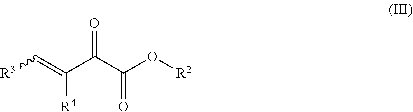

and converting the compound of Formula (III) to the compound of Formula (I).

In some embodiments, the disclosure relates to a method of preparing a compound of Formula (III) comprising coupling a compound of Formula (IV) with a vinyl Grignard reagent of Formula (A).

In some embodiments, R¹ is methyl.

In some embodiments, each R² is chosen from methyl, ethyl, and isopropyl. In some embodiments, each R² is isopropyl.

In some embodiments, R³ and R⁴ are each H.

In some embodiments, the compound of Formula (I) is the compound of Formula (I-A):

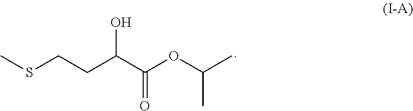

In some embodiments, the compound of Formula (III) is the compound of Formula (III-A):

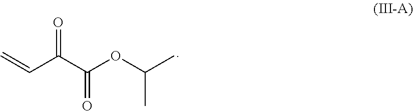

In some embodiments, the vinyl Grignard reagent of Formula (A) is vinyl-MgCl. In some embodiments, X is Cl. In some embodiments, the Grignard coupling is performed in the presence of a salt additive, such as LiCl or ZnCl₂. In some embodiments, the salt additive is LiCl.

In some embodiments, the coupling comprises mixing the compound of Formula (I) with from about 0.8 to about 2.0, or from about 1.0 to about 1.75, or from about 1.0 to about 1.5, or from about 1.2 to about 1.75, or from about 1.4 to about 1.6, or about 1.5 molar equivalents of the vinyl Grignard reagent of Formula (A).

In some embodiments, the coupling is performed at a temperature ranging from about −80° C. to about 10° C., or from about −80° C. to about −70° C., or from about −50 ° C. to about 10° C., or from about −40° C. to about 5° C., or from about −50° C. to about −20° C., or from about −30° C. to about −20° C., or at a temperature of about −78° C., or about −20° C., or about 0° C. In some embodiments, the coupling comprises mixing the compound of Formula (I) in MTBE with about 1.5 molar equivalents of the vinyl Grignard reagent of Formula (A) at a temperature of from about −50° C. to about −20° C., or from about −30° C. to about −20° C. In some embodiments, the vinyl Grignard reagent is added to the compound of Formula (IV) slowly and/or in portions.

In some embodiments, the coupling is performed in an aprotic solvent. In some embodiments, the aprotic solvent is an ether, such as MTBE, THF, or Et₂O, optionally admixed with a non-polar solvent such as heptane or hexanes. In some embodiments, the aprotic solvent is MTBE or THF, optionally admixed with heptane. In some embodiments, the coupling reaction concentration is from about 0.25 M to about 1.3 M (moles of the compound of Formula (IV) per liter of reaction solvent), or from about 0.4 M to about 1.1 M, or from about 0.4 M to about 0.5 M, or from about 0.9 M to about 1.0 M, or about 0.5 M, or about 1 M.

In some embodiments, the coupling produces a mixture of the compound of Formula (III) and a compound of Formula (III-Z):

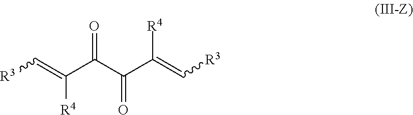

at a ratio of (III):(III-Z) of at least 5:1, or at least 6:1, or at least 7:1, or at least 8:1, or at least 9:1, or at least 10:1, or at least 15:1, or at least 20:1.

In some embodiments, converting the compound of Formula (III) to the compound of Formula (I) comprises:

thiolating the compound of Formula (III) with a thiolating reagent of Formula (B) or of Formula (C):

R¹—SH (B)

R¹—S⁻M⁺ (C)

wherein M⁺ is a metal cation;
to form a compound of Formula (II):

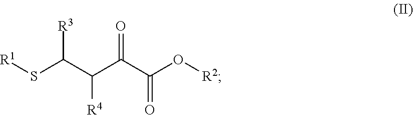

and reducing the compound of Formula (II) to form the compound of Formula (I).

In some embodiments, the disclosure relates to a method of preparing a compound of Formula (II) comprising thiolating a compound of Formula (III) with a thiolating reagent of Formula (B) or of Formula (C).

In some embodiments, the thiolating is performed with the reagent of Formula (B) in the presence of an additive. In some embodiments, the additive is an amine base, such as triethylamine, diethylamine, pentylamine, or hexylamine, a phosphine such as dimethylphenylphosphine (DMPP) or tris(2-carboxyethyl)phosphine (TCEP), a basic salt such as NaHCO₃ or Na₂CO₃, a Lewis acid such as scandium (III) triflate, or anhydrous cerium (III) chloride, an N-heterocyclic carbene (NHC) complex (e.g., an Au-NHC complex). In some embodiments, the additive is triethylamine.

In some embodiments, the method further comprises generating the thiolating reagent of Formula (B) from the thiolating reagent of Formula (C). In some embodiments, the generating is performed in the presence of an acid catalyst. In some embodiments, the acid catalyst is acetic acid, p-toluenesulfonic acid, or H₂SO₄. In some embodiments, the thiolating is performed at a temperature ranging from about −40° C. to about 10° C., or from about −35° C. to about 5° C., or from about −30° C. to about −20° C., or at about 0° C.

In some embodiments, the thiolating agent is Formula (C) and the thiolating is performed at a temperature ranging from about −80° C. to about 35° C., or from about 15° C. to about 35° C.

In some embodiments, M⁺ is Na⁺ or K⁺.

In some embodiments, the coupling comprises extracting the compound of Formula (III) into an organic solvent, to form a Formula (III) extract, and the thiolating comprises adding the thiolating reagent to the Formula (III) extract. In this manner, the thiolation reaction is performed without purifying the intermediate of Formula (III) from the coupling reaction prior to the thiolating reaction. In some embodiments, the procedure is as shown below.

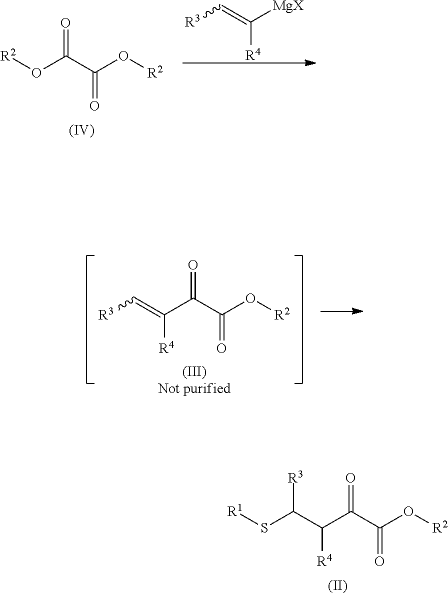

In some embodiments, reducing the compound of Formula (II) is performed in the presence of a reducing agent chosen from NaBH₄, LiBH₄, and Al(O-iPr)₃/iPrOH. In some embodiments, the reducing agent is NaBH₄. In some embodiments, the thiolating comprises extracting the compound of Formula (II) into an organic solvent to form a Formula (II) extract, and the reducing comprises added the reducing agent to the Formula (II) extract. In this manner, the reducing is performed without purifying the compound of Formula (II) prior to the reducing. In some embodiments, the coupling comprises extracting the compound of Formula (III) into an organic solvent to form a Formula (III) extract, the thiolating comprises adding the thiolating reagent to the Formula (III) extract and extracting the compound of Formula (II) into an organic solvent to form a Formula (II) extract, and the reducing comprises adding the reducing agent to the Formula (II) extract. In this manner, the coupling, thiolating, and reducing are performed without purifying the intermediates of Formula (II) and Formula (III), as shown in the following scheme.

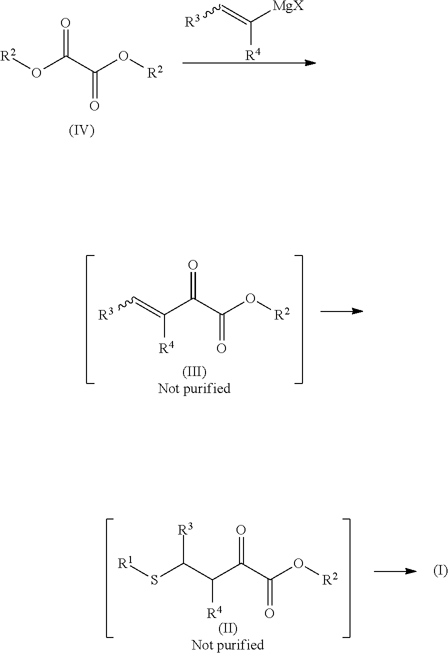

In some embodiments, the reducing is performed with NaBH₄ or LiBH₄:
(a) in an alcohol solvent such as methanol, ethanol, or isopropanol; and/or
(b) using from about 0.25 to about 1.0 molar equivalent of reducing agent; and/or
(c) at a temperature in the range from about −10° C. to about 30° C., or at about 0° C.

In some embodiments, the reducing is performed with Al(O-iPr)₃/iPrOH at a temperature in the range from about 50° C. to about 90° C., or at about 80° C.

In some embodiments, the method further comprises esterifying oxalyl chloride with R²—OH to form the compound of Formula (IV). In some embodiments, the esterifying is performed in the presence of at least one amine base, such as N,N-dimethylpyridine, pyridine, or triethylamine. In some embodiments, the esterifying is performed at a temperature ranging from about −5° C. to about 30° C.

In some embodiments, the method further comprises esterifying oxalic acid with R²—OH in the presence of an acid catalyst and an optional desiccant, such as azeotropic water removal, molecular sieves, or a combination thereof, to form the compound of Formula (IV). In some embodiments, the acid catalyst is chosen from p-TsOH; H₂SO₄; a macroporous sulfonic acid resin catalyst such as Amberlyst®-15, Dowex®, or M32; a silico-aluminate; phosphoric acid; boronic acid; acetyl chloride; and an acid with a pKa below 3. In some embodiments, the acid catalyst is p-TsOH or H₂SO₄. In some embodiments, the acid catalyst is about 0.01 to about 0.1 molar equivalents, or about 0.025 to about 0.05 molar equivalents, of p-TsOH, or about 1 to about 3 molar equivalents, or about 2 molar equivalents, of H₂SO₄. In some embodiments, the esterifying is performed at the reflux temperature of the reaction solvent. In some embodiments, the esterifying is performed in a reaction solvent chosen from toluene, CHCl₃, and isopropanol.

In some embodiments, the disclosure relates to a method of preparing a compound of Formula (I):

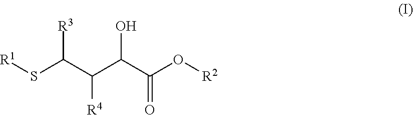

wherein
R¹ is C₁₋₄ alkyl; and
R² is C₁₋₈ alkyl or C₄₋₇ cycloalkyl; and
R³ and R⁴ are each independently chosen from H, methyl, and ethyl;
comprising reducing a compound of Formula (II):

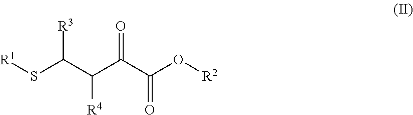

with a reducing agent to form the compound of Formula (I). In some embodiments, the compound of Formula (I) is the compound of Formula (I-A). In some embodiments, the compound of Formula (II) is the compound of Formula (II-A):

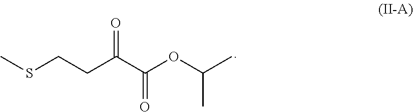

In some embodiments, reducing the compound of Formula (II) is performed in the presence of a reducing agent chosen from NaBH₄, LiBH₄, and Al(O-iPr)₃/iPrOH. In some embodiments, the reducing agent is NaBH₄.

In some embodiments, the reducing is performed with NaBH₄ or LiBH₄:
(a) in an alcohol solvent such as methanol, ethanol, or isopropanol; and/or
(b) using from about 0.25 to about 1.0 molar equivalent of reducing agent; and/or
(c) at a temperature in the range from about −10° C. to about 30° C., or at about 0° C.

In some embodiments, the reducing is performed with Al(O-iPr)₃/iPrOH at a temperature in the range from about 50° C. to about 90° C., or at about 80° C.

In some embodiments, the method further comprises thiolating a compound of Formula (III):

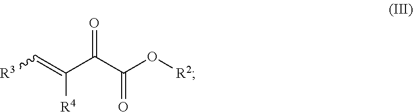

wherein R³ and R⁴ are each independently chosen from H, methyl, and ethyl;
with a thiolating reagent of Formula (B) or of Formula (C):

R¹—SH (B)

R¹—S⁻M⁺ (C)

wherein M⁺ is a metal cation;
to form the compound of Formula (II). In some embodiments, the compound of Formula (II) is the compound of Formula (II-A) and the compound of Formula (III) is the compound of Formula (III-A):

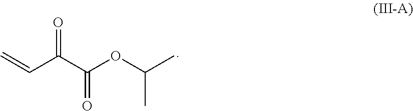

In some embodiments, the disclosure relates to a method of preparing a compound of Formula (I-A):

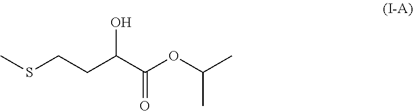

comprising:
esterifying oxalic acid with isopropanol to form diisopropyl oxalate;
coupling diisopropyl oxalate with vinylmagnesium bromide to form a compound of Formula (III-A):

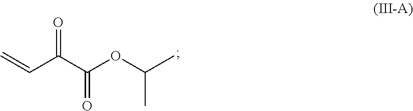

thiolating the compound of Formula (III-A) with CH₃SH to form a compound of Formula (II-A):

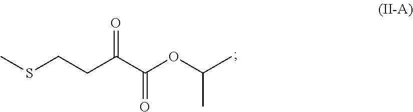

and reducing the compound of Formula (II-A) to form the compound of Formula (I-A).

In some embodiments, the methods described herein provide the compound of Formula (I) or Formula (I-A) in at least about 95% purity by GC, HPLC, and/or by weight. In some embodiments, the methods provide a crude compound of Formula (I) or Formula (I-A) that has a purity by weight, GC, and/or HPLC of at least about 95%, or at least about 96%, or at least about 97%, or at least about 98%, wherein the crude compound of Formula (I) or Formula (I-A) has not been purified or has been purified only by fractional distillation. In some embodiments, the methods provide a crude compound of Formula (I) or Formula (I-A) that is substantially in monomeric form, or that comprises less than about 5% by weight, or less than about 3%, by weight, of dimeric and/or oligomeric compounds, wherein the crude compound of Formula (I) or Formula (I-A) has not been purified or has been purified only by fractional distillation.

### Compound Products

In some embodiments, the reacting provides a crude compound of Formula (I) or Formula (I-A) that has a purity by weight (and/or by GC or HPLC) of at least about 80%, or at least about 90%, or at least about 95%, or at least about 96%, or at least about 97%, or at least about 98%, wherein the crude compound of Formula (I) or Formula (I-A) has not been purified or has been purified only by fractional distillation. In some embodiments, the reacting provides a crude compound of Formula (I) or Formula (I-A) that is substantially in monomeric form, or that comprises less than 5% by weight, or less than 3%, by weight, of dimeric and/or oligomeric compounds, wherein the crude compound of Formula (I) or Formula (I-A) has not been purified or has been purified only by fractional distillation.

In some embodiments, the disclosure relates to a compound of Formula (I) or Formula (I-A) prepared as in a method described herein. In some embodiments, the disclosure relates to a compound of Formula (I) or Formula (I-A), wherein the compound has a purity by weight (and/or by GC or HPLC) of at least about 80%, or at least about 90%, or at least about 95%, or at least about 96%, or at least about 97%, or at least about 98%, and the compound has not been purified or has been purified only by fractional distillation. In some embodiments, the compound is substantially in monomeric form, or is mixed with less than about 5%, or less than about 3%, by weight, of dimeric and/or oligomeric compounds.

In some embodiments, the HMBi (Formula (I-A)) product has one or more of the following specifications: (a) at least about 95% by weight or by HPLC analysis HMBi monomer content and chemical purity; and (b) water content of less than about 0.5% by Karl Fischer analysis; (c) pH less than about 6.0 (measured at 1% concentration in water).

Also disclosed herein is compound of Formula (I) or Formula (I-A) prepared by any of the methods described herein. In some embodiments is a compound of Formula (I) or Formula (I-A), wherein the compound has a purity by weight (and/or by GC or HPLC) of at least about 95%, or at least about 96%, or at least about 97%, or at least about 98%, and the compound is a crude compound, has not been purified, and/or has been purified only by fractional distillation. In some embodiments, the compound is the compound of Formula (I), wherein R¹ is —CH₂CH₂—S—CH₃ and R² is isopropyl, or the compound is the compound of Formula (I-A). In some embodiments, the compound is substantially in monomeric form, or is mixed with less than about 5%, or less than about 3%, by weight, of dimeric and/or oligomeric compounds.

### Animal Feed Compositions and Uses

In some aspects, the present disclosure relates to an animal feed composition comprising the compound of Formula (I) or Formula (I-A) as described herein. In some embodiments, animal feed composition is suitable for administration to ruminants, such as cattle, cows, sheep, antelope, deer, giraffes, bovines (e.g., bison, buffalo, or yak), goats, and/or gazelles. In some embodiments, the animal feed composition is a cow feed composition, such as a dairy cow feed composition, or an additive for cow feed, such as dairy cow feed. In some embodiments, the animal feed composition is a dairy cow feed composition.

In some embodiments, the animal feed composition is an animal feed or an animal feed additive. In some embodiments, the animal feed additive is in liquid or solid form, wherein the liquid form comprises the compound and optionally a liquid carrier, and the solid form comprises the compound admixed with a solid carrier, optionally wherein the solid carrier is silica (silicon dioxide), optionally wherein the ratio of the compound to solid carrier is from about 5:1 to about 1:5, or is 3:2. In some embodiments, the feed composition is liquid feed additive or a solid feed additive. In some embodiments, the animal feed composition is drinking water additive. In some embodiments, the liquid feed additive or drinking water additive has a pH ranging from about 4.0 to about 7.5.

In some embodiments of the animal feed composition, R¹ is —CH₂CH₂—S—CH₃ and R² is isopropyl. In some embodiments, the compound is the compound of Formula (I-A).

In some embodiments, the disclosure relates to a method of supplying bioavailable methionine to a dairy cow comprising administering to the cow the compound or animal feed composition described herein. In some embodiments, administering comprises feeding to the cow a feed composition containing the compound. In some embodiments, the disclosure relates to a method of supplying at least about 50% bioavailable methionine to a dairy cow comprising administering to the cow the compound or animal feed composition as described herein. In some embodiments, the disclosure relates to a method of improving milk obtained from a dairy cow, comprising supplying to the cow the compound or animal feed composition as described herein. In some embodiments, the improvement in the milk comprises increased protein content in the milk. In some embodiments, the improvement in the milk comprises increased fat content in the milk. In some embodiments, the disclosure relates to a method of improving the condition of a cow comprising supplying to the cow the compound or animal feed composition as described herein. In some embodiments, the improvement in the condition of the cow comprises improved fertility. In some embodiments, the improvement in the condition of the cow comprises improved liver function. In some embodiments, the improvement in the condition of the cow comprises an increase in energy.

In some aspects, any of the reactions described herein may be performed using a continuous flow apparatus.

### EXAMPLES

Equipment. All mmol-scale experiments were carried out using a 100 mL or 250 mL three-neck round-bottom flask with a magnetic stirrer, a dropping funnel, and a thermometer. The reaction flask was equipped with a condenser and a thermometer to monitor the reaction temperature. For reactions run at reflux, the reaction mixture was heated using a silicon oil-bath. For experiments at temperatures below room temperature, a liquid nitrogen bath or salt/ice mixture bath was used. All kg-scale experiments were carried out using a 5 L jacketed reactor. The concentration and/or purification of intermediates and crude products were carried out using a laboratory scale vacuum distillation unit, a rotary evaporator, or column chromatography, or as otherwise specified in the following examples.

### Example 1. Synthesis of Diisopropyl Oxalate from Oxalic Acid

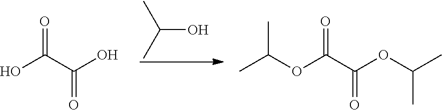

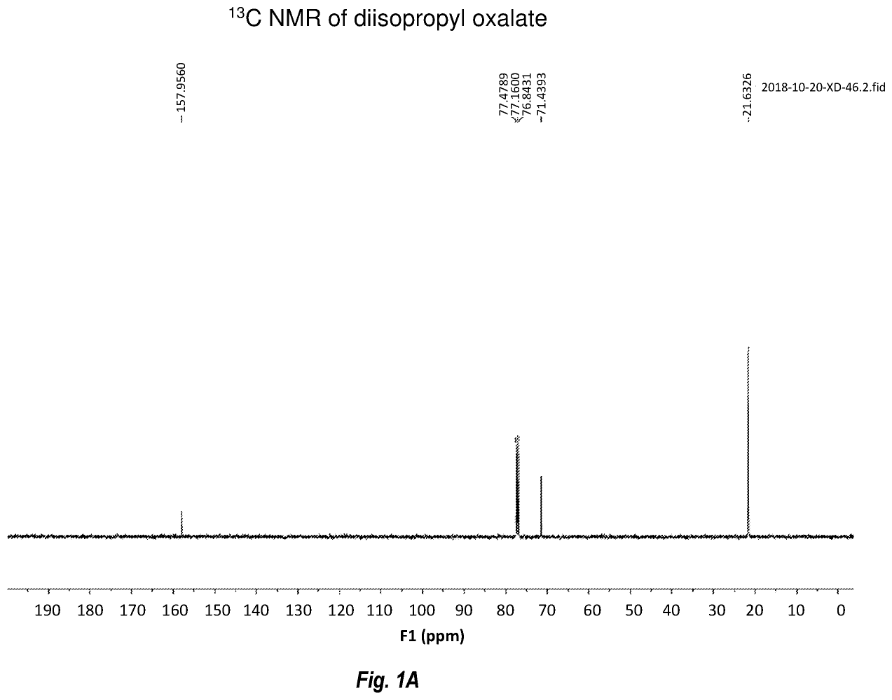

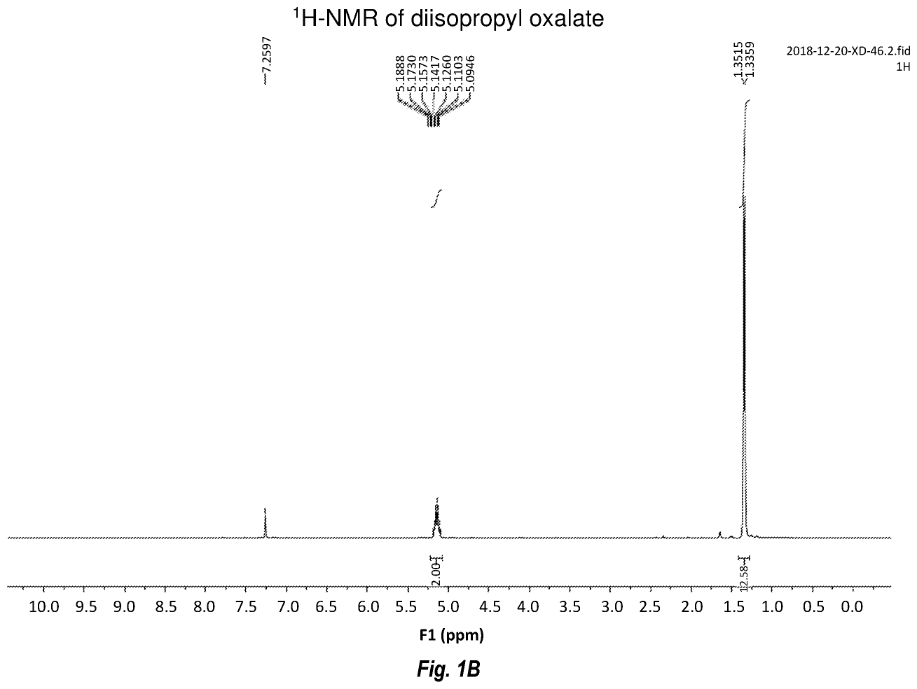

Oxalic acid (1 kg, 11.1 mol) was added to isopropyl alcohol (1700 mL) under stirring in a 5 L laboratory reactor. A clear solution formed. Subsequently, p-toluenesulfonic acid monohydrate (47.67 g, 2.5 mol %) in 200 mL toluene was slowly added to the solution. The reaction mixture was heated up and stirred a reflux for 24 h. The generated water was continuously removed by azeotrope with a Dean-Stark trap to drive the reaction to completion. The reaction mixture was cooled down, neutralized with 500 mL satd. aq. NaHCO₃, and partitioned between with 400 mL (2×) toluene and 1 L (2×) water. The combined organic phases were dried with 1 L of satd. aq. NaCl solution. The organic phase was separated, and the solvent was removed under vacuum. The crude material was purified by distillation under high vacuum with heating to obtain 1740 g (90%) diisopropyl oxalate as colorless oil. ¹³C NMR (100 MHz, CDCl₃) δ (ppm) 157.96, 71.44, 21.63 (FIG. 1A); ¹H NMR (400 MHz, CDCl₃) δ5.13 (hept, J=6.3 Hz, 2 H), 1.33 (d, J=6.2 Hz, 12 H) (FIG. 1B).

Various other suitable reaction conditions were investigated, as shown in Table 1, using oxalic acid dihydrate (entries 1-5) or oxalic acid (entries 6-7) as the starting material. To the reaction mixture, 4 Å molecular sieves (1-2 g per 5 g of starting material) were added to remove additional water during the reaction. The work-up included diluting the reaction mixture with ethyl acetate, neutralizing to pH 7 with said. aq. NaHCO₃, separating the layers, washing the organic extract with satd. aq. NaHCO3 and satd. aq. NaCl, and concentrating to provide a crude residue.

TABLE 1

| | Concentration | | | |
|---|---|---|---|---|
| | (g oxalic | | | |
| | acid/mL | Other Solvent | | |
| Entry | iPrOH) | (volume) | Additive | Yield |
| 1 | 5 g/12 mL | CHCl₃ (40 mL) | Conc. H₂SO₄ (2.0 eq.) | 68% |
| 2 | 50 g/65 mL | Toluene (400 mL) | None | 47% |
| 3 | 50 g/65 mL | Toluene (400 mL) | p-TsOH (0.05 eq.) | 61% |
| 4 | 50 g/65 mL | None | Conc. H₂SO₄ (2.0 eq.) | 51% |
| 5 | 50 g/65 mL | None | p-TsOH (0.05 eq.) | 69% |
| 6 | 50 g/100 mL | Toluene (400 mL) | p-TsOH (0.05 eq.) | 90% |
| 7 | 25 g/50 mL | Toluene (200 mL) | None | 79% |

### Example 2. Synthesis of Diisopropyl Oxalate from Oxalyl Chloride

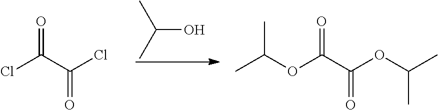

To a 0° C. sample of isopropyl alcohol (3 L) in a 5 L glass lined laboratory reactor was added oxalyl chloride (1019 g) slowly in portions with stirring while maintaining the temperature between 0 and 5° C. After the addition was complete, the reaction mixture was allowed to gradually warm up to room temperature and was stirred for 12 h. The mixture was concentrated by rotary evaporation and high vacuum to obtain the crude product. The crude product was diluted with dichloromethane (1000 mL) and washed with said. aq. NaHCO₃ (3×500 mL) to provide an organic extract. The first two aqueous washes were back-extracted with dichloromethane (1 L each) to provide two additional organic extracts. The three organic extracts were dried with satd. aq. NaCl (3×500 mL), combined, concentrated, and purified by distillation to obtain diisopropyl oxalate in 86% yield. ¹H NMR (400 MHz, CDCl₃) δ5.13 (hept, J=6.3 Hz, 2 H), 1.33 (d, J=6.2 Hz, 12 H).

Various other suitable reaction conditions were investigated, as shown in Table 2.

TABLE 2

| | Concentration | | | |
|---|---|---|---|---|
| | (g oxalyl | | | |
| | chloride/mL | | | |
| Entry | iPrOH) | Additive | Work-Up Conditions | Yield |
| 1 | 7.6 g/30 mL | None | Concentrate under | 92% |
| | | | vacuum | |
| 2 | 3.8 g/4.6 mL | 4.7 g pyridine, | Column | 54% |
| | | 183 mg DMAP | chromatography | |
| 3 | 25.4 g/100 mL | None | CH₂Cl₂/NaHCO₃ | 85% |
| | | | extraction; concentrate | |
| | | | under vacuum | |
| 4 | 100 g/300 mL | None | Concentrate under | 81% |
| | | | vacuum | |
| 5 | 1019 g/3000 mL | None | Purified by distillation | 86% |

### Example 3. Synthesis of 2-Oxo-4-methylthiobutanoic Acid Isopropyl Ester (Small-Scale Experiments)

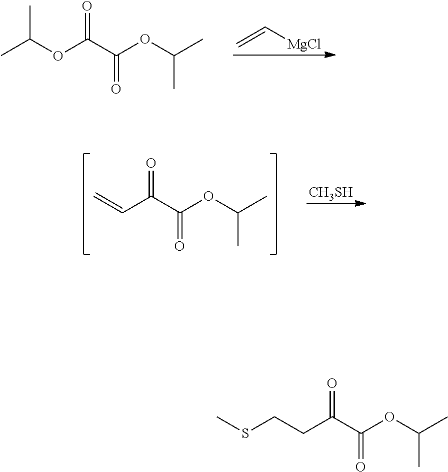

Step 1, Grignard Reaction. A mixture of diisopropyl oxalate (1.4 g, 8 mmol, 1.0 eq.), 16 mL solvent (MTBE, MTBE/heptane mixture, or THF) and 2 eq. LiCl (when used, 0.68 g, 16 mmol) was cooled down to the test temperature (as shown in Table 3) either under a liquid nitrogen bath or salt bath. A solution of vinyl magnesium chloride (1.6 M in THF) was slowly added and the resulting mixture was stirred until the starting material is consumed (see Table 3). The reaction was quenched by washing with satd. aq. NH₄Cl (2×100 mL). The product was extracted with EtOAc (2×100 mL), dried over Na₂SO₄, and filtered. The yield of 2-oxo-3-butenoic acid isopropyl ester was determined by GC/MS. The extract was used directly in the next step without purification.

TABLE 3

| | VinylMgCl (equiv.); | Temperature; | | Conver- | |
|---|---|---|---|---|---|
| Entry | Solvent | Reaction Time | Additive | sion | Yield |
| 1 | 1.0; THF | −78° C.; 4 h | None | 68% | 58% |
| 2 | 1.0; THF | −20° C.; 4 h | None | 50% | 38% |
| 3 | 1.0; THF | 0° C.; 2 h | None | 44% | 21% |
| 4 | 1.0; MTBE | −78° C.; 1 h | None | 73% | 61% |
| 5 | 1.0; MTBE/heptane | −78° C.; 1 h | None | 73% | 62% |
| 6 | 1.5; THF | −78° C.; 2 h | None | 96% | 71% |
| 7 | 1.5; THF* | −78° C.; 2 h | None | 83% | 56% |
| 8 | 1.5; MTBE | −78° C.; | None | 100% | 80% |
| | | 30 min | | | |
| 9 | 1.5; MTBE | −78° C.; 1 h | LiCl | 100% | 85% |
| 10 | 1.0; THF | −78° C.; 1 h | LiCl | 71% | 59% |
| 11 | 1.5; THF | −78° C.; 1 h | LiCl | 100% | 80% |
| 12 | 1.5; MTBE | −30 to −20° C.; | None | 100% | 72% |
| | | 30 min | | | |
| 13 | 1.5; THF | −30 to −20° C.; | None | 100% | 78% |
| | | 30 min | | | |
| 14 | 1.5; MTBE | −30 to −20° C.; | None | ND | ND |
| | | 1 h | | | |
| 15 | 1.5; MTBE | −30 to −20° C.; | None | ND | ND |
| | | 15 min | | | |

*Diisopropyl oxalate added to Grignard reagent in this example.
ND = not determined

Step 2, Thiolation Reaction.

Procedure 1: CH₃SH gas was generated by treating 20% w/v CH₃SNa in water with acid catalyst (AcOH (12 mmol) or TsOH (12 mmol)) at −30 to −20° C., or with H₂SO₄ (2 equiv. relative to CH₃SNa) at 50° C.) for 15 to 30 min as shown in Table 4. The resulting CH₃SH was bubbled into a stirred 0° C. solution of MTBE (20 mL) containing triethylamine (0.1 mL). The resulting CH₃SH in MTBE was added to the crude product in MTBE from Step 1, Table 3, Entry 14, at 0° C., or at −30 to −20° C., and the reaction mixture was stirred from 15 to 30 min, as indicated in Table 4. The reaction was quenched with 2 M HCl, extracted with ethyl acetate, dried (Na₂SO₄), filtered, and concentrated. The crude material was then used for the next reaction step.

In Entries 1-5 in Table 4, acetic acid or p-toluenesulfonic acid were used to generate CH₃SH gas. In Entries 1-3, the yield is the isolated yield after column chromatography. In Entries 4-5, the yield is the isolated yield after product distillation. In Entries 6-9, CH₃SH gas was generated by heating 20% CH₃SNa in water and H₂SO₄ at 50° C.

TABLE 4

| | | Temperature; | Yield |
|---|---|---|---|
| Entry | Acid | Reaction Time | (2-step) |
| 1 | p-toluenesulfonic acid | 0° C; 30 min | 27% |
| 2 | p-toluenesulfonic acid | −30 to −20° C.; 15 min | 46% |
| 3 | acetic acid | −30 to −20° C.; 15 min | 38% |
| 4 | p-toluenesulfonic acid | −30 to −20° C.; 15 min | 49% |
| 5 | acetic acid | −30 to −20° C.; 15 min | 44% |
| 6 | H₂SO₄ | −30 to −20° C.; 15 min | 20% |
| 7 | H₂SO₄ | −30 to −20° C.; 15 min | 19% |
| 8 | H₂SO₄ | −30 to −20° C.; 15 min | 54% |
| 9 | H₂SO₄ | −30 to −20° C.; 15 min | 63% |

Procedure 2: To the crude product from Step 1, Table 3, Entry 11, in THF at −78° C. was added 20% aq. w/v CH₃SNa (1 eq.) and H₂SO₄ (2 eq.). The reaction mixture was allowed to warm to room temperature and was stirred for 16 h. The reaction was quenched with 2 M HCl, extracted with ethyl acetate (2×50 mL), dried (Na₂SO₄), filtered, and concentrated. The crude was then used for the next reaction step. The product was isolated to provide the product in 33% yield. ¹H NMR (400 MHz, CDCl₃) δ5.12 (hept, J=6.2 Hz, 1 H), 3.13 (t, J=7.2 Hz, 2 H), 2.76 (t, J=7.2 Hz, 2 H), 2.11 (s, 3 H), 1.33 (d, J=6.3 Hz, 6 H).

Procedure 3, Continuous Flow Reactor: Alternatively, a mixture of 2-oxo-3-butenoic acid isopropyl ester and 10 mL of triethylamine is charged into a reactor via a pump with a controlled flowrate. The outlet is further connected to an inlet of a Y-mixer, where another inlet is charged by MeSH gas (with a control flow). The two components are then mixed and further stirred within a batch reactor while maintaining the reaction temperature at 0° C. Once GC monitoring indicates reaction completion, 1 N HCl is added and the mixture is worked up as described above.

### Example 4: Synthesis of 2-Oxo-4-methylthiobutanoic Acid Isopropyl Ester (Kilogram Scale Synthesis)

Step 1, Grignard Reaction. To a −30 to −20° C. stirring solution of diisopropyl oxalate (1.7 kg, 10 mol) in anhydrous MTBE (3.4 L) in a 20 L laboratory reactor was added vinyl magnesium chloride (1.6 M in THF, 7 L) dropwise over a period of 1 h, maintaining the temperature at −30° C. to −20° C. Once gas chromatography indicated completion of the vinyl addition, the reaction was quenched by the addition of 1 L satd. aq. NH₄Cl at room temperature. The organic phase was separated and washed with 500 mL water, and the aqueous phase was back-extracted with 400 mL MTBE. The MTBE extracts were combined to provide 2-oxo-3-butenoic acid isopropyl ester in greater than 90% conversion, which was used directly in the next step without further purification or distillation.

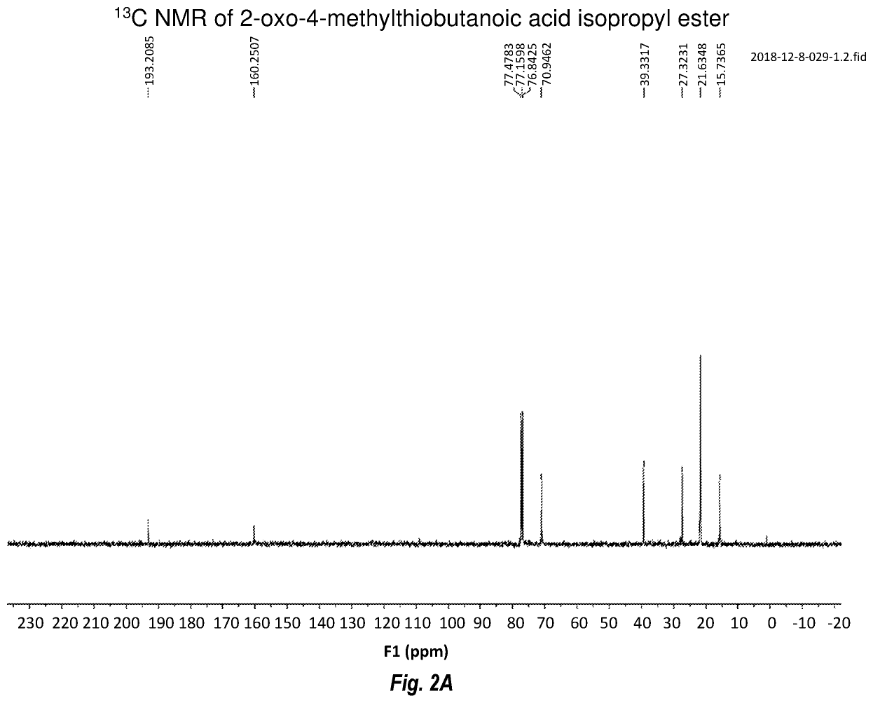

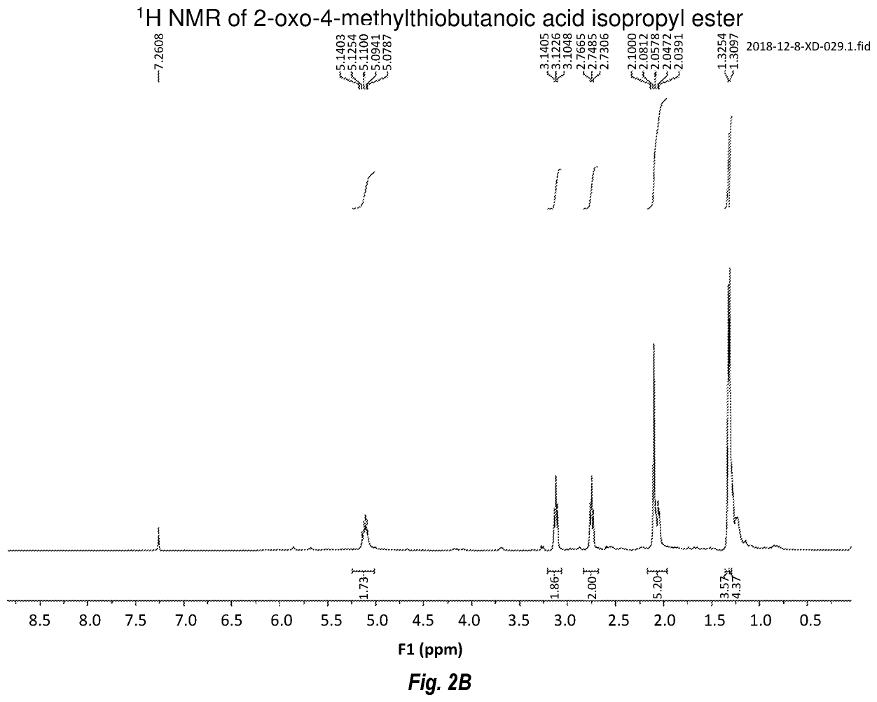

Step 2, Thiolation. The MTBE extract from Step 1 was cooled to 0° C. in the reactor and was treated with triethylamine (10 mL). MeSH gas was generated in situ by reacting a solution of CH₃SNa (1.0 eq.; 20% in water) with H₂SO₄ (2 equiv.) at 50° C., for 30 min. The resulting CH₃SH gas was bubbled into the stirred 0° C. reaction solution, and stirring was continued at 0° C. When GC monitoring indicated conversion of the intermediate to 2-oxo-4-methylthiobutanoic acid isopropyl ester, 1 N HCl (780 mL) was added to the reactor to quench the reaction. The organic layer was separated from the aqueous phase, washed with 500 mL water, and the aqueous phase was extracted with 650 mL (2×) MTBE. The combined organic extracts were concentrated by vacuum evaporation and the product was purified by distillation under vacuum to obtain 1021 g (55%) of 2-oxo-4-methylthiobutanoic acid isopropyl ester as colorless oil. ¹³C NMR (100 MHz, CDCl₃) δ (ppm) 193.21, 160.25, 70.95, 39.33, 27.32, 21.63, 15.74 (FIG. 2A); ¹H NMR (400 MHz, CDCl₃) δ5.12 (hept, J=6.2 Hz, 1 H), 3.13 (t, J=7.2 Hz, 2 H), 2.76 (t, J=7.2 Hz, 2 H), 2.11 (s, 3 H), 1.33 (d, J=6.3 Hz, 6 H) (FIG. 2B).

### Example 5: Synthesis of 2-Hydroxy-4-methylthiobutanoic Isopropyl Ester (HMBi)

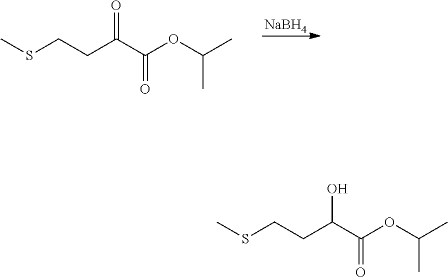

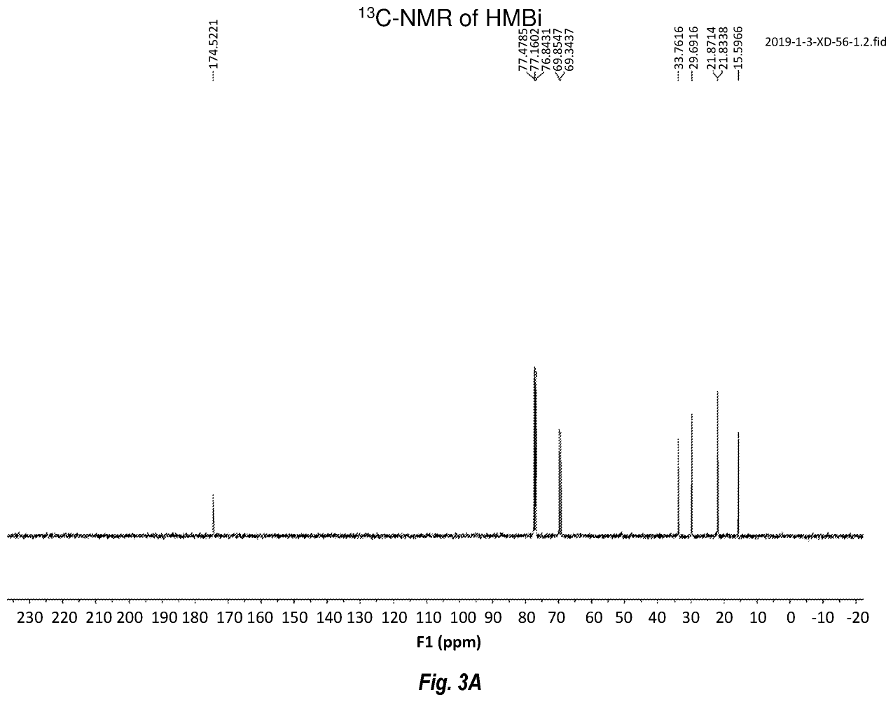

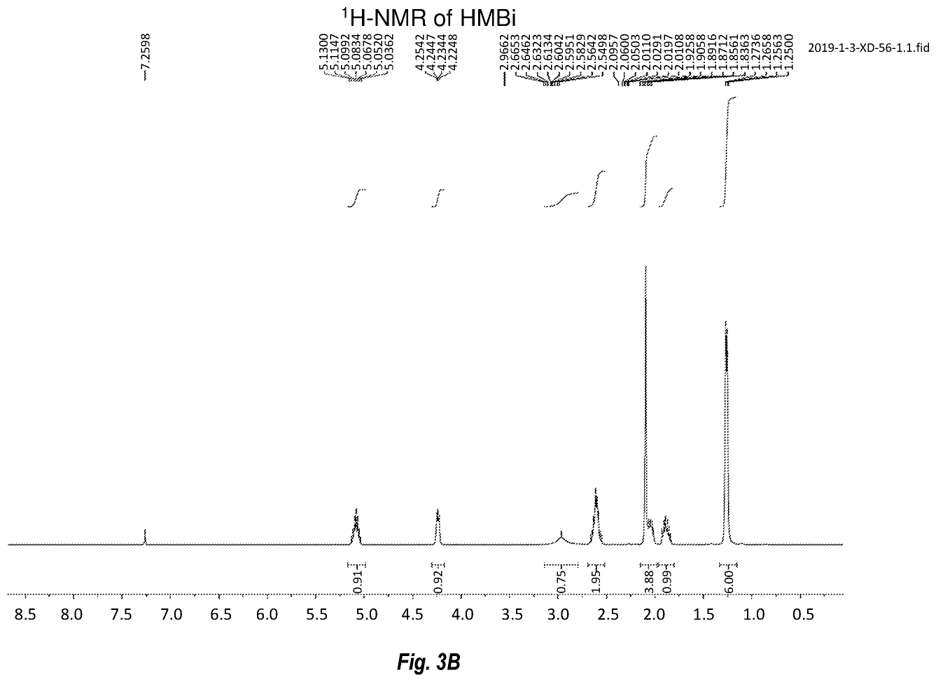

To a 0 to 5° C. solution of 2-oxo-4-methylthiobutanoic acid isopropyl ester (1 kg, 5.25 mol) in methanol (2 L) in a 5 L reactor was added NaBH₄ (99 g, 2.6 mol) in portions. The resulting reaction was maintained between 0 to 5° C. and was stirred for 1 h. The reaction mixture was washed with satd. aq. NH₄Cl (500 mL). The organic phase was separated, the solvent was removed by vacuum distillation, and the crude product was purified by distillation to give HMBi (859 g, 85% yield, 97% monomeric ester) as a pale yellow oil. ¹³C NMR (100 MHz, CDCl₃) δ (ppm) 174.52, 69.85, 69.34, 33.76, 29.69, 21.87, 21.83, 15.60. (FIG. 3A); ¹H NMR (400 MHz, CDCl₃) δ5.08 (hept, J=6.3 Hz, 1 H), 4.24 (dd, J=7.9, 3.8 Hz, 1 H), 2.97 (br, 1 H), 2.67-2.55 (m, 2 H), 2.10-2.01 (m, 4 H), 1.93-1.84 (m, 1 H), 1.27 (d, J=1.9 Hz, 3 H), 1.26 (d, J=2.2 Hz, 3 H) (FIG. 3B).

Alternative for the syntheses of 2-hydroxy-4-(methylthio) butanoic isopropyl ester (HMBi) from 2-oxo-4-methylthiobutanoic isopropyl ester (OMBi):

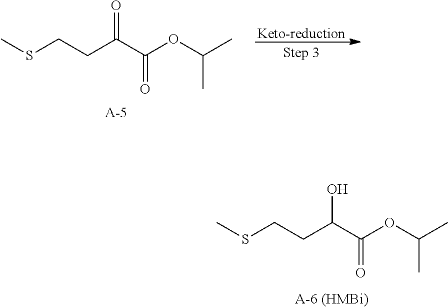

Various reagents and conditions, including NaBH₄, transition-metal catalyzed hydrogenation, and keto-reduction were screened for the preparation of HMBi from 2-oxo-4-methylthiobutanoic acid isopropyl ester. Several reaction temperatures, times, reagents and solvents were tested. The product conversion and the yield of the isolated product from each set of conditions were determined and the results are shown in Table 5.

TABLE 5

| Entry | Conditions | Yield |
|---|---|---|
| 1 | NaBH₄ (1 eq.), EtOH, 0° C., 1 h | 30% |
| 2 | NaBH₄ (0.25 eq.), EtOH, 0° C., 1 h | 72% |
| 3 | 5 mol % Pd (Pd/C), H₂ (1 atm), i-PrOH, | no reaction |
| | 25° C., 1 h | occurred |
| 4 | 5 mol % Pd (Pd/C), H₂ (1 atm), toluene, | no reaction |
| | 25° C., 1 h | occurred |
| 5 | 5 mol % Pd (Pd/C), H₂ (1 atm), MTBE, | no reaction |
| | 50° C., 1 h | occurred |
| 6 | 5 mol % Pd (Pd/C), H₂ (1 atm), THF, | no reaction |
| | 50° C., 1 h | occurred |
| 7 | 2.5 mol % [Ru(p-cymene) Cl₂]₂, H₂ | no reaction |
| | (1 atm), i-PrOH, 75° C., 1 h | occurred |
| 8 | 5 mol % CpRu(PPh₃)₂Cl, H₂ (1 atm), | no reaction |
| | i-PrOH, 75° C., 1 h | occurred |
| 9 | NaBH₄ (0.25 eq), i-PrOH, 0° C., 1 h | 27% |
| 10 | Al (OᵢPr)₃ (1 eq), i-PrOH, 80° C., N₂ | 28% |
| | bubble to remove acetone, 1 h | |
| 11 | NaBH₄ (0.5 eq.), EtOH, 0° C., 1 h | 73% |
| 12 | NaBH₄ (0.5 eq.), MeOH, 0° C., 1 h | 92% |

1. A method of preparing a compound of Formula (I): 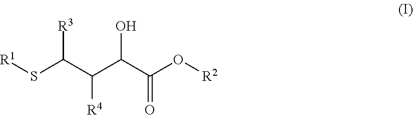
wherein
R¹ is C₁₋₄ alkyl; and
R² is C₁₋₈ alkyl or C₄₋₇ cycloalkyl; and
R³ and R⁴ are each independently chosen from H, methyl, and ethyl;
comprising coupling a compound of Formula (IV): 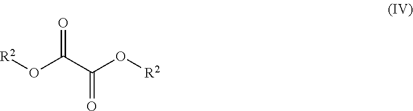
with a vinyl Grignard reagent of Formula (A): 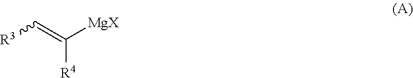
wherein X is Br or Cl;
to form a compound of Formula (III): 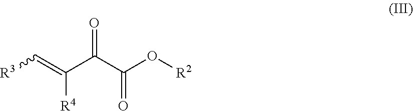
and converting the compound of Formula (III) to the compound of Formula (I).

2. The method of claim 1, wherein R¹ is methyl.

3. The method of claim 1 or claim 2, wherein each R² is chosen from methyl, ethyl, and isopropyl.

4. The method of claim 3, wherein each R² is isopropyl.

5. The method of any one of claims 1 to 4, wherein R³ and R⁴ are each H.

6. The method of claim 1, wherein the compound of Formula (I) is the compound of Formula (I-A):

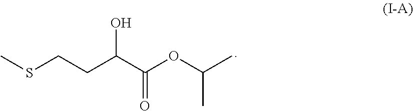

7. The method of claim 1, wherein the compound of Formula (III) is the compound of Formula (III-A):

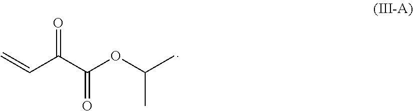

8. The method of any one of claims 1 to 7, wherein the vinyl Grignard reagent of Formula (C) is vinyl-MgCl.

9. The method of any one of claims 1 to 7, wherein X is Cl.

10. The method of claim 9, wherein the coupling is performed in the presence of a salt additive, such as LiCl or ZnCl₂.

11. The method of any one of claims 1 to 10, wherein the coupling comprises mixing the compound of Formula (I) with from about 0.8 to about 2.0, or from about 1.0 to about 1.75, or from about 1.0 to about 1.5, or from about 1.2 to about 1.75, or from about 1.4 to about 1.6, or about 1.5 molar equivalents of the vinyl Grignard reagent of Formula (A).

12. The method of any one of claims 1 to 11, wherein the coupling is performed at a temperature ranging from about −80° C. to about 10° C., or from about −80° C. to about −70° C., or from about −50° C. to about 10° C., or from about −40° C. to about 5° C., or from about −50° C. to about −20° C., or from about −30° C. to about −20° C., or at a temperature of about −78° C., or about −20° C., or about 0° C.

13. The method of any one of claims 1 to 12, wherein the coupling is performed in an aprotic solvent.

14. The method of claim 13, wherein the aprotic solvent is an ether, such as MTBE, THF, or Et₂O, optionally admixed with a non-polar solvent such as heptane or hexanes.

15. The method of claim 14, wherein the aprotic solvent is MTBE or THF, optionally admixed with heptane.

16. The method of any one of claims 1 to 15, wherein the coupling reaction concentration is from about 0.25 M to about 1.3 M (moles of the compound of Formula (IV) per liter of reaction solvent), or from about 0.4 M to about 1.1 M, or from about 0.4 M to about 0.5 M, or from about 0.9 M to about 1.0 M, or about 0.5 M, or about 1 M.

17. The method of any one of claims 1 to 12, wherein the coupling produces a mixture of the compound of Formula (III) and a compound of Formula (III-Z): 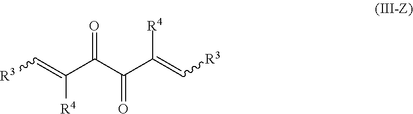
at a ratio of (III):(III-Z) of at least 5:1, or at least 6:1, or at least 7:1, or at least 8:1, or at least 9:1, or at least 10:1, or at least 15:1, or at least 20:1.

18. The method of any one of claims 1 to 17, wherein converting the compound of Formula (III) to the compound of Formula (I) comprises:
thiolating the compound of Formula (III) with a thiolating reagent of Formula (B) or of Formula (C):
R¹—SH (B)
R¹—S⁻M⁺ (C)
wherein M⁺ is a metal cation;
to form a compound of Formula (II): 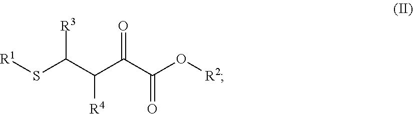
and reducing the compound of Formula (II) to form the compound of Formula (I).

19. The method of claim 18, wherein the thiolating is performed with the reagent of Formula (B) in the presence of an additive.

20. The method of claim 19, wherein the additive is an amine base, such as triethylamine, diethylamine, pentylamine, or hexylamine, a phosphine such as dimethylphenylphosphine (DMPP) or tris(2-carboxyethyl)phosphine (TCEP), a basic salt such as NaHCO₃ or Na₂CO₃, a Lewis acid such as scandium (III) triflate, or anhydrous cerium (III) chloride, or an N-heterocyclic carbene (NHC) complex such as an Au-NHC complex.

21. The method of claim 19, wherein the additive is triethylamine.

22. The method of any one of claims 18 to 21, further comprising generating the thiolating reagent of Formula (B) from the thiolating reagent of Formula (C).

23. The method of claim 22, wherein the generating is performed in the presence of an acid catalyst.

24. The method of claim 23, wherein the acid catalyst is acetic acid, p-toluenesulfonic acid, or H₂SO₄.

25. The method of any one of claims 22 to 24, wherein the thiolating is performed at a temperature ranging from about −40° C. to about 10° C., or from about −35° C. to about 5° C., or from about −30° C. to about −20° C., or at about 0° C.

26. The method of any one of claims 18 to 21, wherein the thiolating agent is Formula (C) and the thiolating is performed at a temperature ranging from about −80° C. to about 35° C., or from about 15° C. to about 35° C.

27. The method of any one of claims 18 to 26, wherein M⁺ is Na⁺ or K⁺.

28. The method of any one of claims 18 to 27, comprising extracting the compound of Formula (III) into an organic solvent to form a Formula (III) extract, and thiolating the compound of Formula (III) comprises adding the thiolating reagent to the Formula (III) extract.

29. The method of any one of claims 18 to 28, wherein reducing the compound of Formula (II) is performed in the presence of a reducing agent chosen from NaBH₄, LiBH₄, and Al(O-iPr)₃/iPrOH.

30. The method of claim 29, wherein the reducing agent is NaBH₄.

31. The method of any one of claims 18 to 30, wherein reducing is performed:
(a) in an alcohol solvent such as methanol, ethanol, or isopropanol; and/or
(b) using from about 0.25 to about 1.0 molar equivalent of reducing agent; and/or
(c) at a temperature in the range from about −10° C. to about 30° C., or at about 0° C. when the reducing agent is not Al(O-iPr)₃/iPrOH, or when the reducing agent is Al(O-iPr)₃/iPrOH from about 50° C. to about 90° C., or at about 80° C.

32. The method of any one of claims 18 to 31, wherein the thiolating comprises extracting the compound of Formula (II) into an organic solvent to form a Formula (II) extract, and the reducing comprises added the reducing agent to the Formula (II) extract.

33. The method of any one of claims 1 to 32, further comprising esterifying oxalyl chloride with R²—OH to form the compound of Formula (IV).

34. The method of claim 33, wherein the esterifying is performed in the presence of at least one amine base, such as N,N-dimethylpyridine, pyridine, or triethylamine.

35. The method of claim 33 or claim 34, wherein the esterifying is performed at a temperature ranging from about −5° C. to about 30° C.

36. The method of any one of claims 1 to 33, further comprising esterifying oxalic acid with R²—OH in the presence of an acid catalyst and an optional desiccant, such as azeotropic water removal, molecular sieves, or a combination thereof, to form the compound of Formula (IV).

37. The method of claim 36, wherein the acid catalyst is chosen from p-TsOH; H₂SO₄; a macroporous sulfonic acid resin catalyst such as Amberlyst®-15, Dowex®, or M32; a silico-aluminate; phosphoric acid; boronic acid; acetyl chloride; and an acid with a pKa below 3.

38. The method of claim 36, wherein the acid catalyst is p-TsOH or H₂SO₄.

39. The method of claim 36, wherein the acid catalyst is about 0.01 to about 0.1 molar equivalents, or about 0.025 to about 0.05 molar equivalents, of p-TsOH, or about 1 to about 3 molar equivalents, or about 2 molar equivalents, of H₂SO₄.

40. The method of any one of claims 36 to 39, wherein the esterifying is performed at the reflux temperature of the reaction solvent.

41. The method of any one of claims 33 to 40, wherein the esterifying is performed in a reaction solvent chosen from toluene, CHCl₃, and isopropanol.

42. A method of preparing a compound of Formula (I): 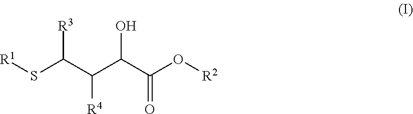
wherein
R¹ is C₁₋₄ alkyl; and
R² is C₁₋₈ alkyl or C₄₋₇ cycloalkyl; and
R³ and R⁴ are each independently chosen from H, methyl, and ethyl;
comprising reducing a compound of Formula (II): 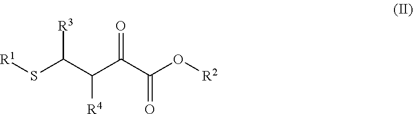
with a reducing agent to form the compound of Formula (I).

43. The method of claim 42, wherein R¹ is methyl.

44. The method of claim 42 or claim 43, wherein each R² is chosen from methyl, ethyl, and isopropyl.

45. The method of claim 44, wherein each R² is isopropyl.

46. The method of any one of claims 42 to 45, wherein R³ and R⁴ are each H.

47. The method of any claim 42, wherein the compound of Formula (I) is the compound of Formula (I-A):

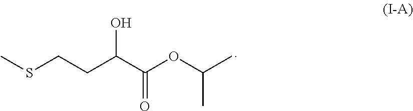

48. The method of any one of claims 42 to 47, wherein the reducing agent is chosen from NaBH₄, LiBH₄, and Al(O-iPr)₃/iPrOH.

49. The method of claim 48, wherein the reducing agent is NaBH₄.

50. The method of any one of claims 42 to 49, wherein reducing is performed:
(a) in an alcohol solvent such as methanol, ethanol, or isopropanol; and/or
(b) using from about 0.25 to about 1.0 molar equivalent of reducing agent; and/or
(c) at a temperature in the range from about −10° C. to about 30° C., or at about 0° C. when the reducing agent is not Al(O-iPr)₃/iPrOH, or when the reducing agent is Al(O-iPr)₃/iPrOH from about 50° C. to about 90° C., or at about 80° C.

51. The method of any one of claims 42 to 49, further comprising thiolating a compound of Formula (III): 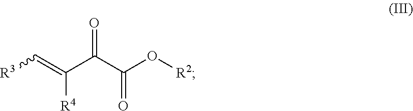
wherein R³ and R⁴ are each independently chosen from H, methyl, and ethyl;
with a thiolating reagent of Formula (B) or Formula (C):
R¹—SH (B)
R¹—S⁻M⁺ (C)
wherein M⁺ is a metal cation;
to form the compound of Formula (II).

52. The method of claim 51, wherein the compound of Formula (III) is the compound of Formula (III-A):

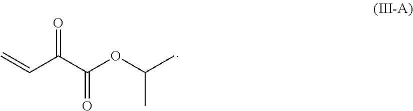

53. A method of preparing a compound of Formula (I-A): 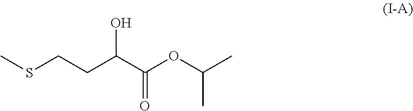
comprising:
esterifying oxalic acid with isopropanol to form diisopropyl oxalate;
coupling diisopropyl oxalate with vinylmagnesium bromide to form a compound of Formula (III-A): 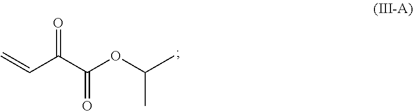
thiolating the compound of Formula (III-A) with CH₃SH to form a compound of Formula (II-A): 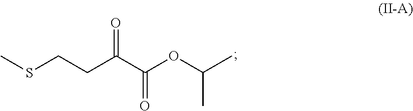
and reducing the compound of Formula (II-A) to form the compound of Formula (I-A).

54. The method of any one of claims 1 to 53, wherein the method provides the compound of Formula (I) or Formula (I-A) in at least about 95% purity by GC, HPLC, and/or by weight.

55. The method of any one of claims 1 to 54, wherein the method provides a crude compound of Formula (I) or Formula (I-A) that has a purity by weight, GC, and/or HPLC of at least about 95%, or at least about 96%, or at least about 97%, or at least about 98%, wherein the crude compound of Formula (I) or Formula (I-A) has not been purified or has been purified only by fractional distillation.

56. The method of any one of claims 1 to 55, wherein the method provides a crude compound of Formula (I) or Formula (I-A) that is substantially in monomeric form, or that comprises less than about 5% by weight, or less than about 3%, by weight, of dimeric and/or oligomeric compounds, wherein the crude compound of Formula (I) or Formula (I-A) has not been purified or has been purified only by fractional distillation.

57. A compound of Formula (I) or Formula (I-A) prepared by the method of any one of claims 1 to 56.

58. An animal feed composition comprising the compound of claim 57.

59. The animal feed composition of claim 58, wherein the animal feed composition is a cow feed composition, such as a dairy cow feed composition, or an additive for cow feed, such as dairy cow feed.

60. The animal feed composition of claim 59, wherein the animal feed composition is a dairy cow feed composition.

61. The animal feed composition of any one of claims 58 to 60, wherein the animal feed composition is an animal feed or an animal feed additive.

62. The animal feed composition of claim 61, wherein the animal feed additive is in liquid or solid form, wherein the liquid form comprises the compound and optionally a liquid carrier, and the solid form comprises the compound admixed with a solid carrier, optionally wherein the solid carrier is silica (silicon dioxide), optionally wherein the ratio of the compound to solid carrier is from about 5:1 to about 1:5, or is about 3:2.

63. A method of supplying bioavailable methionine to a dairy cow comprising administering to the cow the compound of claim 57 or animal feed composition of any one of claims 58 to 62.

64. A method of supplying at least about 50% bioavailable methionine to a dairy cow comprising administering to the cow the compound of claim 57 or animal feed composition of any one of claims 58 to 62.

65. A method of improving milk obtained from a dairy cow, comprising supplying to the cow the compound of claim 57 or animal feed composition of any one of claims 58 to 62.

66. The method of claim 65, wherein the improvement in the milk comprises increased protein content in the milk.

67. The method of claim 65, wherein the improvement in the milk comprises increased fat content in the milk.

68. A compound of claim 57 or animal feed composition of any one of claims 58 to 62 for use in a method of improving milk obtained from a dairy cow.

69. The compound or composition for use of claim 68, wherein the improvement in the milk comprises increased protein content in the milk.

70. The compound of composition for use of claim 68, wherein the improvement in the milk comprises increased fat content in the milk.

71. A method of improving the condition of a cow comprising supplying to the cow the compound of claim 57 or animal feed composition of any one of claims 58 to 62.

72. The method of claim 71, wherein the improvement in the condition of the cow comprises improved fertility.

73. The method of claim 71, wherein the improvement in the condition of the cow comprises improved liver function.

74. The method of claim 71, wherein the improvement in the condition of the cow comprises an increase in energy.

75. The method of any one of claims 63 to 67 or 71 to 74, wherein administering or supplying comprises feeding to the cow the animal feed composition.

76. A compound of claim 57 or animal feed composition of any one of claims 58 to 62 for use in a method of improving the condition of a cow.

77. The compound or composition for use of claim 76, wherein the improvement in the condition of the cow comprises improved fertility.

78. The compound or composition for use of claim 76, wherein the improvement in the condition of the cow comprises improved liver function.

79. The compound or composition for use of claim 76, wherein the improvement in the condition of the cow comprises an increase in energy.

80. A compound that is isopropyl 2-oxobut-3-enoate.
